(19) United States
(12) Patent Application Publication
(10) Pub. No.: US 2009/0306667 A1
(43) Pub. Date: Dec. 10, 2009

(54) METHOD AND APPARATUS FOR CERVICAL FUSION

(76) Inventors: David LEE, Hattiesburg, MS (US); James Payne, JR., Vancleave, MS (US)

Correspondence Address: GARVEY SMITH NEHRBASS & NORTH, LLC, LAKEWAY 3, SUITE 3290, 3838 NORTH CAUSEWAY BLVD., METAIRIE, LA, 70002, US

(21) Appl. No.: 12/411,433

(22) Filed: Mar. 26, 2009

Related U.S. Application Data
(60) Provisional application No. 61/039,486, filed on Mar. 26, 2008.

(51) Int. Cl. A61B 17/80; A61B 17/88

(52) U.S. Cl. 606/70; 606/281

(57) ABSTRACT

A cervical spinal fixation system that offers a strong and stable construct for maximum fusion augmentation and yet is versatile enough for any patient and is easy to use. A method of implanting a fusion plate is disclosed where the plate includes at least two primary fastener openings. In one embodiment the method includes the step of the throat of the patient being opened which provides access to the spinal column of the patient. The fusion plate can be inserted into the throat opening, and the fusion plate is then positioned on the spinal column.

### CROSS-REFERENCE TO RELATED APPLICATIONS

Priority of U.S. Provisional Patent Application Ser. No. 61/039,486, filed Mar. 26, 2008, incorporated herein by reference, is hereby claimed.

### BACKGROUND

The human spinal column includes a row of 26 bones or vertebrae in the back and allows a person to stand up straight and bend over. The spinal column also protects a person's spinal cord from being hurt. The vertebrae are commonly classified by position into cervical (neck), thoracic (chest), and lower back (lumbar) vertebrae, with intervertebral discs separating each vertebra from the adjacent vertebrae.

In the neck, degeneration of discs often causes vertebrae to undesirably compress their associated spinal nerves, causing uncomfortable symptoms such as pain, numbness, weakness, and disordered reflex symptoms. Degenerated cervical discs may be treated by resection of the discs utilizing a surgical approach through the neck. Once the resection is completed, common procedure is to fuse the vertebrae adjoining the former position of the removed disc. Other conditions which may require fusion include treatment of fractured or broken vertebrae, correction of deformities, treatment of herniated discs, treatment of tumors, treatment of infections, or treatment of instability.

Fusion is a surgical technique in which one or more of the vertebrae of the spine are united or joined to prevent relative movement. The spinal fusion procedure does not directly connect the vertebrae; rather, a bone graft or spacer is positioned between endplates of adjacent vertebrae of the spine during surgery. Over a period of time healing occurs as living bone from vertebrae spans the intervertebral graft and connects the adjacent vertebrae together. Fusion is complete when living bone has completely spanned the graft and the adjacent vertebrae are thus connected by a solid bridge of bone.

Various apparatus are known for retaining vertebrae of a spinal column in a desired spatial relationship so that fusion of the vertebrae can occur. Such known apparatus can include rod or plate systems, with either commonly being attached to the vertebrae with bone screws, hooks, or other structures. For example, anterior fusion of the cervical spine is commonly stabilized using a fixation plate screwed to the vertebrae. The rods and/or plates can be temporary (removed after fusion of the vertebrae is complete) or permanent.

However, currently available plates are generally larger than needed to simply maintain adjacent cervical vertebrae in fixed orientation in most patients. These oversized structures require a relatively large incision and dissection for insertion, which may be complex and time-consuming for the surgeon and require broader dissection of tissues in the neck and greater pressure on tissues being moved out of the way. This generally results in longer healing time and a larger risk of complications in the patient than in a smaller incision and dissection.

Accordingly, it is desired to develop procedures and implants for surgically addressing stenosis through minimally invasive procedures, and preferably such surgical procedures can be performed on an outpatient basis.

### SUMMARY

In one embodiment is provided a method of implanting a fusion plate, having at least two openings, into a patient is described.

In one embodiment of the method, a throat of the patient is dissected, providing access through the throat dissection to a spinal column of the patient. The fusion plate is inserted into the throat dissection, and the fusion plate is then positioned in on the spinal column.

In one embodiment a portion of an anterior surface of a first vertebra of the spinal column and a portion of an anterior surface of a second vertebra of the spinal column are contacted by the fusion plate.

While certain novel features of this invention shown and described below are pointed out in the annexed claims, the invention is not intended to be limited to the details specified, since a person of ordinary skill in the relevant art will understand that various omissions, modifications, substitutions and/or changes in the forms and details of the device illustrated and in its operation may be made without departing in any way from the spirit of the present invention. No feature of the invention is critical or essential unless it is expressly stated as being “critical” or “essential.”

### FIGURES

FIG. 1 is a top perspective view of one embodiment of a single level cervical fusion plate.

FIG. 2 is a bottom perspective view of the fusion plate of FIG. 1.

FIG. 3 is a bottom perspective view of the fusion plate of FIG. 1.

FIG. 4 is a top perspective view of the fusion plate of FIG. 1 where one of the locking covers is in a locked position and the second locking cover is in an unlocked position.

FIG. 5 is a bottom perspective view of the fusion plate of FIG. 4.

FIG. 6 is a side perspective view of the fusion plate of FIG. 1.

FIG. 7 is a front perspective view of the fusion plate of FIG. 1.

FIG. 8 is a rear perspective view of the fusion plate of FIG. 1.

FIG. 9 is an anterior perspective view of the fusion plate of FIG. 1 installed on two vertebrae.

FIG. 10 is a perspective view from the line 10-10 of FIG. 9.

FIG. 11 is a left side perspective view of the fusion plate of FIG. 9.

FIG. 12 is a right side perspective view of the fusion plate of FIG. 9.

FIG. 13 is a top perspective view of the fusion plate of FIG. 9.

FIG. 14 is another perspective view from the line 10-10 of FIG. 9.

FIG. 15 is a top view of the fusion plate of FIG. 1 where the locking covers have been removed for clarity.

FIG. 16 is a top view of an alternative fusion plate for two levels where the locking covers have been removed for clarity.

FIG. 17 is a top view of an alternative fusion plate for three levels where the locking covers have been removed for clarity.

FIG. 18 is a top view of an alternative fusion plate for four levels where the locking covers have been removed for clarity.

FIG. 19 is a top view of a locking cover.

FIG. 20 is a top view of the fusion plate of FIG. 15 showing the locking covers in an unlocked position.

FIG. 21 is a top view of an alternative fusion plate of FIG. 16 for two levels showing the locking covers in an unlocked position.

FIG. 22 is a top view of an alternative fusion plate of FIG. 17 for three levels showing the locking covers in an unlocked position.

FIG. 23 is a top view of an alternative fusion plate of FIG. 18 for four levels showing the locking covers in an unlocked position.

FIG. 24 is a top view of the fusion plate of FIG. 15 now showing the locking covers in a locked position.

FIG. 25 is a top view of an alternative fusion plate of FIG. 16 for two levels now showing the locking covers in a locked position.

FIG. 26 is a top view of an alternative fusion plate of FIG. 17 for three levels showing the locking covers in a locked position.

FIG. 27 is a top view of an alternative fusion plate of FIG. 18 for four levels showing the locking covers in a locked position.

FIG. 28 is a side view of a installation tool which can be used to lock and unlock the locking covers.

FIG. 29 is a bottom view of the tip of the installation tool

### DETAILED DESCRIPTION

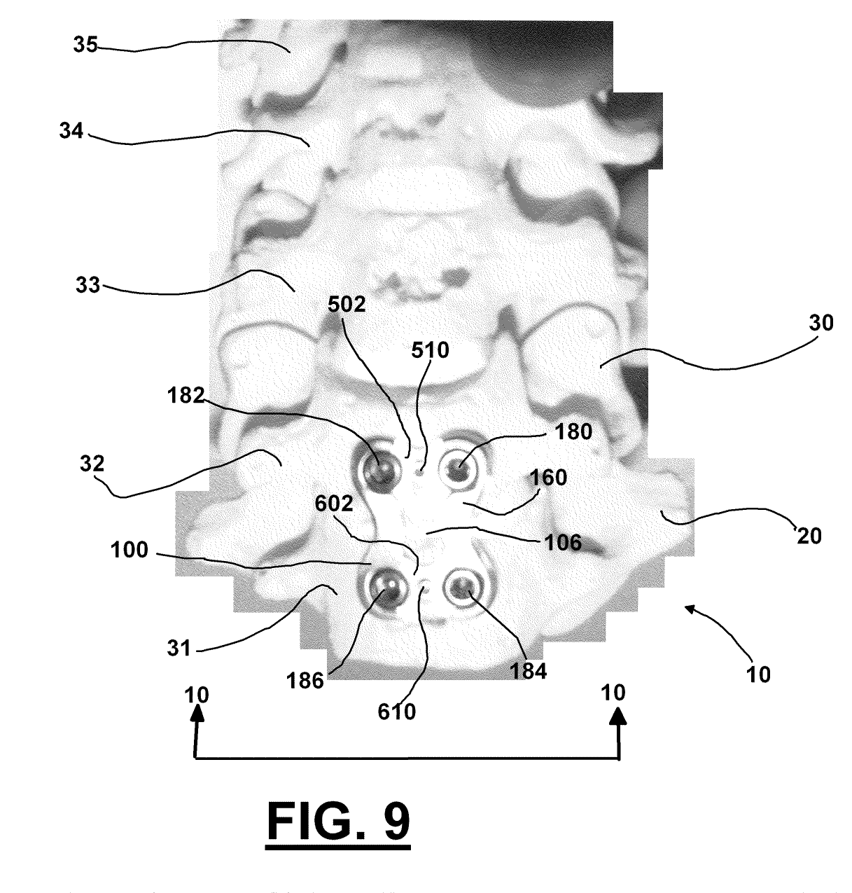

FIG. 9 shows an anterior perspective view of a cervical fusion apparatus 10. The cervical fusion apparatus 100 can include includes a cervical fusion plate 100 which is attached to cervical spinal column 20. Spinal column 20 includes a plurality of vertebrae 30 (such as vertebra 31, 32, 33, 34). The drawings will show fusion plates which can be used for fusing one, two, three, or four levels.

In one embodiment anterior cervical locking plate 100 is provided. Plate 100 can be used for a single level fusion (two adjacent vertebra). Plate 200 can be used for a two level fusion (three adjacent vertebrae). Plate 300 can be used for a three level fusion (four adjacent vertebrae). Plate 400 can be used for a four level fusion (five adjacent vertebrae).

However, the description will describe in detail only the plate 100 for a one level system; as the construction and operation of the fusion plates for additional levels are similar with a repetition of the individual components of the fusion plate for each additional level.

Each vertebra 31, 32, 33, 34, etc., has an anterior surface substantially centered about the midline sagittal plane. Cervical fusion plate 100 can be adapted to simultaneously engage a plurality of vertebrae (e.g., 32 and 33) of the spinal column 20 to retain the engaged vertebrae in a desired relationship (and the engaged vertebrae need not be adjacent). However, in the interest of simplicity only a two level fusion system will be shown installed.

The construction of cervical locking plate 100 will be generally described. Plate 100 can have a generally linear form with an overall shape of a parallelogram (such as a rectangle) with rounded corners. However, additional shapes of cervical fusion plate 100 can be used (such as elliptical, rectangular, triangular, etc.).

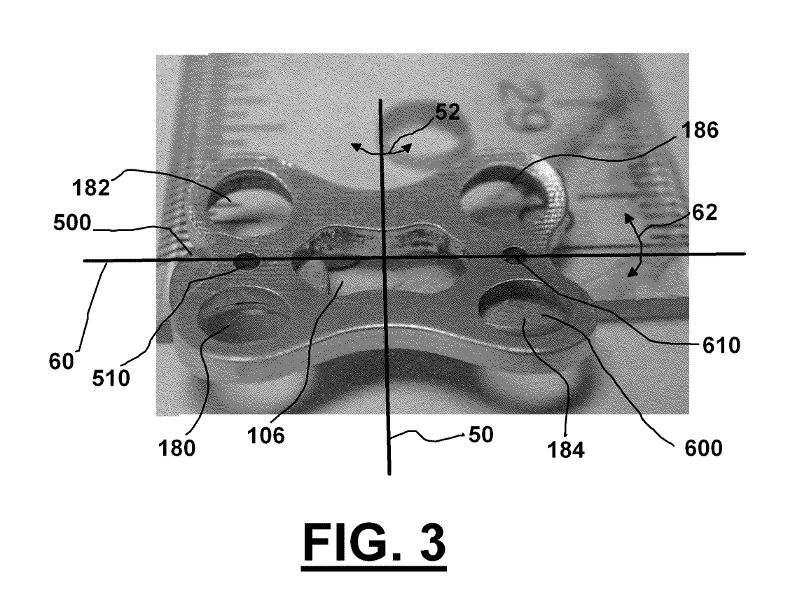

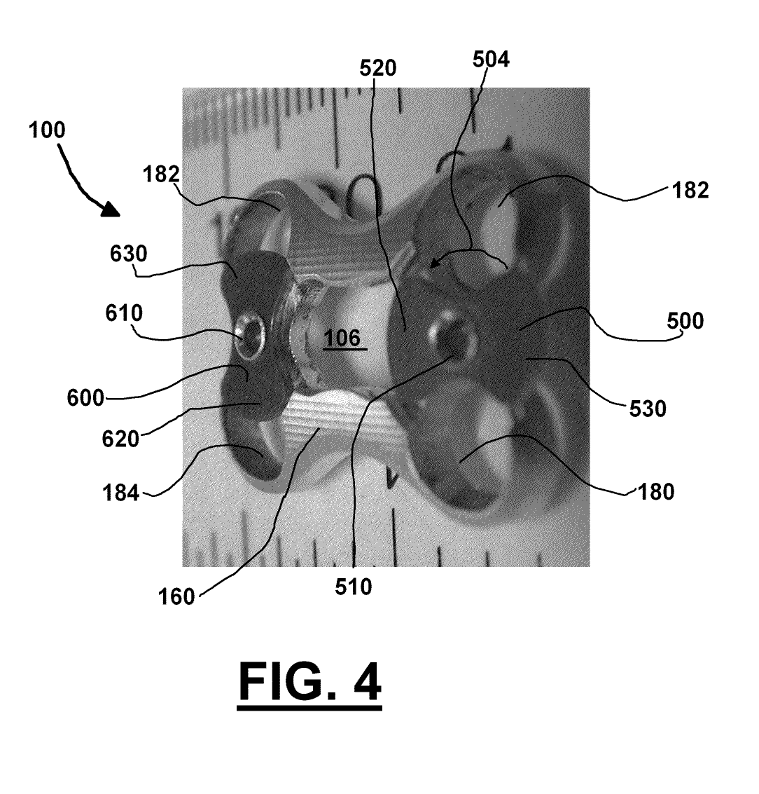

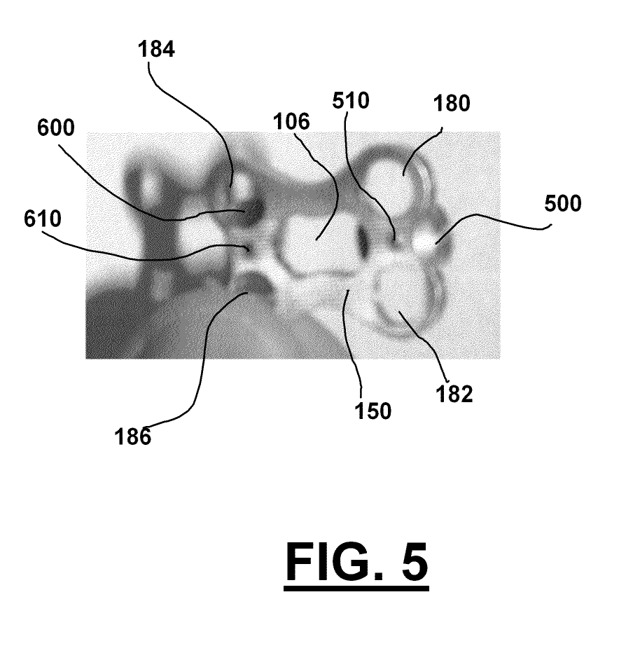

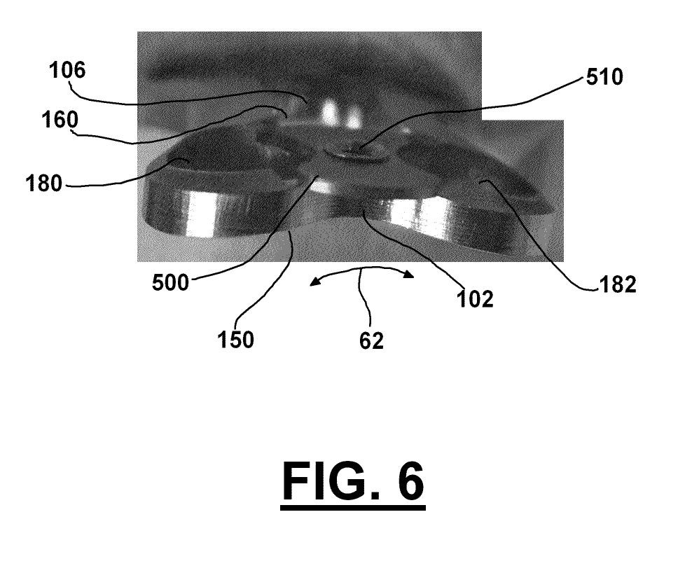

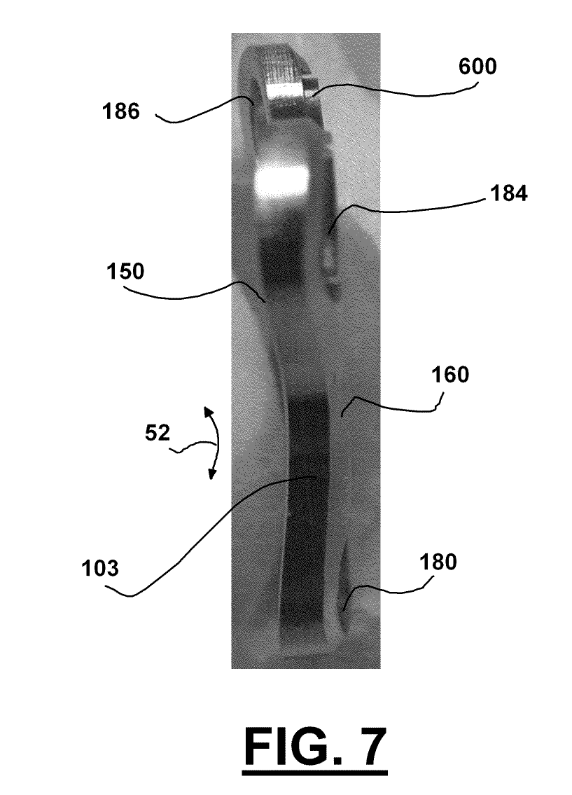

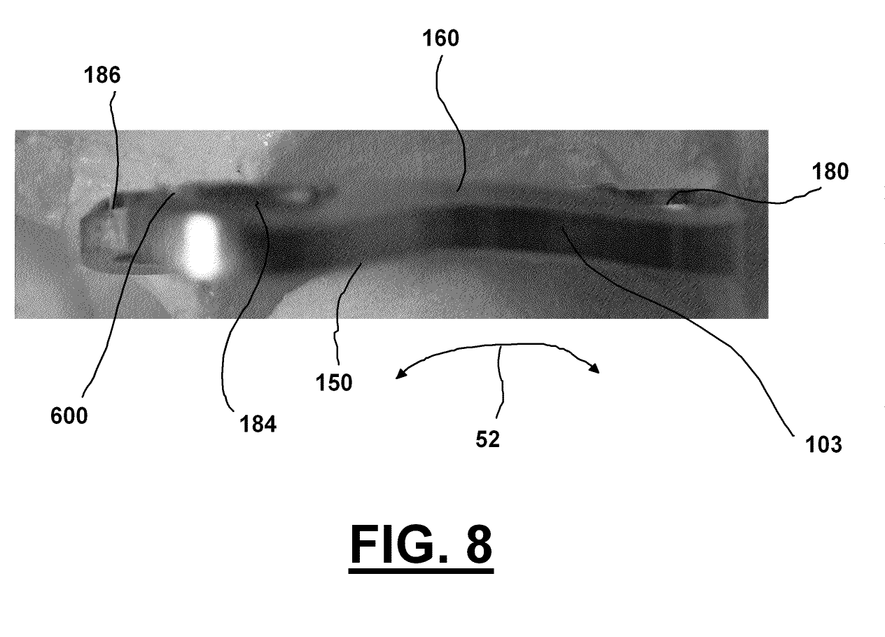

Plate 100 can be curved in both the traverse and sagittal planes. Plate 100 can be shaped so that its bottom surface 150 (the surface which will be in contact with the anterior portion of the vertebrae) has a bi-concave curvature, being concave both in the longitudinal plane (corresponding to its length—see arrow 62 in FIG. 3) and in the plane transverse thereto, corresponding to its width (see arrow 52 in FIG. 3). The concave curvature in the longitudinal plane can conform to the proper shape of the anterior aspect of the spine with the vertebrae aligned in appropriate lordosis. Having plate 100 curved in two orthogonal directions (along its length and its width) allows the connecting bone screws to enter the vertebrae at converging angles, and these converging angles of the bone screws are believed to increase the overall stability of fusion plate 100 and the anchored vertebrae (allowing for a better overall fusion between the anchored vertebrae).

In one embodiment that longitudinal curve can be an arc along the circumference of a circle (referred to herein as the “radius of curvature”) which is typically 15.0 centimeters to 30.0 centimeters in radius, and more preferably 20.0-25.0 centimeters.

In one embodiment, plate 100 can have has a radius of curvature of between 15 to 25 millimeters, preferably between 19-21 millimeters. In one embodiment, plate 100 can have a thickness between 2 to 3 millimeters, preferably between 2.25 and 2.5 millimeters. In one embodiment, the curvature of plate 100 conforming closely to the surface of the anterior cervical spine reduces the amount of projection of plate 100 from the anterior of the spinal column 20, reduces the risk that soft tissue will slide underneath the plate's edges, and reduces the risk of damage.

Various portions of cervical fusion plate 100 can be removed from its overall shape (e.g., portions 102, 103, 104, 105, and 106). Removal of these portions can decrease the overall size of plate 100 along with increasing the opacity of plate 100 to subsequent investigative and/or viewing techniques. Such increased opacity to X-rays or other investigative techniques can allow for better viewing and tracking of the progress of the overall fusion. For example, as will be described below viewing window 106 can be provided in the approximate position of the middle of the two vertebrae to be fused. This window 106 can allow a post surgery investigative technique which can view the overall progress of the fusion such as be looking at the connection point or points between upper and lower vertebrae being fused.

Plate 100 can include connecting fastener openings 180, 182, 184, and 186, which can be circular. These openings can be recessed so that screw heads are flush with the surface of plate 100. Alternatively, these openings may have any suitable configuration, such as substantially round holes, elongated slots, or open-sided notches in fusion plate 100. These openings may be filleted, chamfered, countersunk, or otherwise shaped to accept the corresponding fasteners, respectively, in a snug mating relationship and optionally prevent any portion of the fastener, such its fastener head, from protruding from plate 100 once implantation is complete.

In one embodiment screw openings 180, 182, 184, and 186, can be shaped to guide a screw into a predetermined angular relationship with one or more of the other screws. The angular placements of the screws with respect to the cervical fusion plate 100 and/or the vertebrae may be determined by one of ordinary skill in the art, but should be chosen to engage the plate 100 and the vertebrae together to retain the vertebrae in the desired spatial relationship while accommodating individual patient anatomy. Alternatively, the longitudinal axes the screws can form a converging angle in the transverse plane.

Each screw may be of any suitable type (e.g., bolt, screw, spike, barbed rod, adhesive/peg, or the like). The screws used in any one plate are preferably of the same type and size, however, they need not be matched in type, size, material, position, or any other characteristic and may be readily selected as desired by one of ordinary skill in the art.

Preferably, the screw openings may have anchoring structures associated with such openings. In one embodiment cervical fusion plate 100 can include one or more rotating locking covers or plates 500, 600. Rotating locking covers can be pivotally connected to plate 100 at rotation points 510, 610.

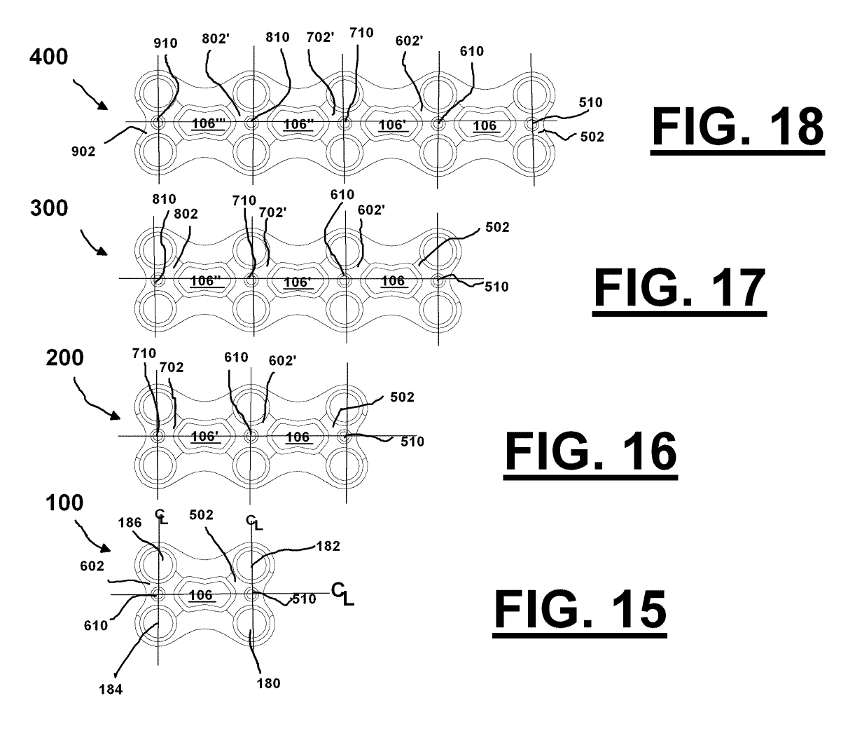

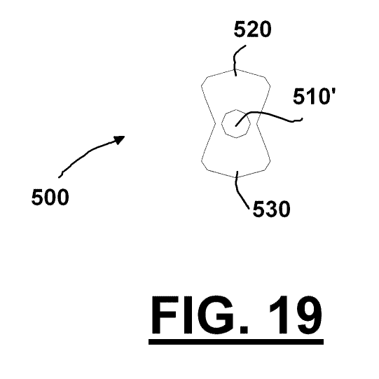

In one embodiment locking covers 500 and 600 can be flushly mounted on plate 100 (respectively in recesses 502 and 602). Locking cover 500 can fit inside recess 106 to enable cover plate 500 to be flush with the top surface of plate 100. Cover plate 500 can include first end 520 and second end 530. As shown in FIG. 15, pivot points 510 and 610 can be located on a centerline respectively between openings 180/182 and 184/186. Additionally, pivot points 510 and 610 can be located on a centerline passing through the longitudinal center of window or cutout 106.

Arrows 504, 604 schematically indicate the ability of locking cover 500 to rotate relative to plate 100. In a first position locking cover 500 does not block a portion of any of the holes such as 180 an 182. However, in a second position locking cover rotates to block a portion of holes 180 and 182. Locking covers 500 and 600 can be pivotally connected to plate 100 such that friction tends to lock or resist movement of covers relative to plate 100 (and a substantial force is required to rotate such locking covers).

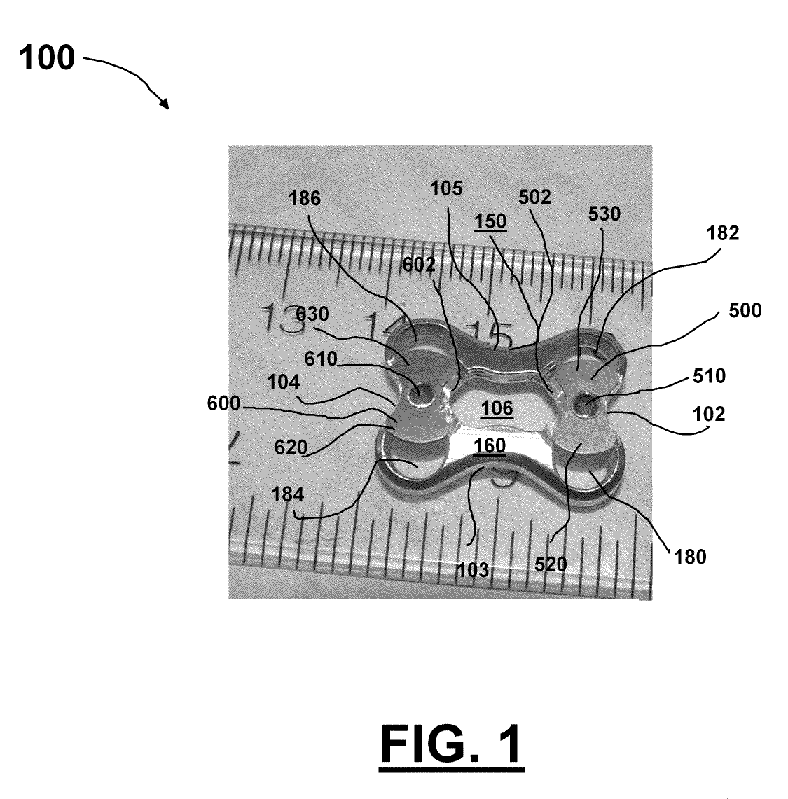

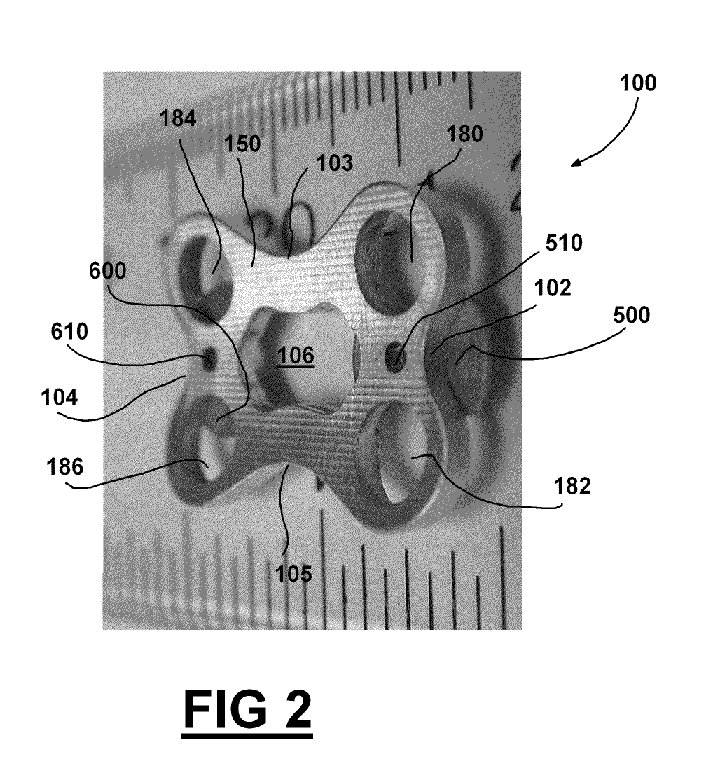

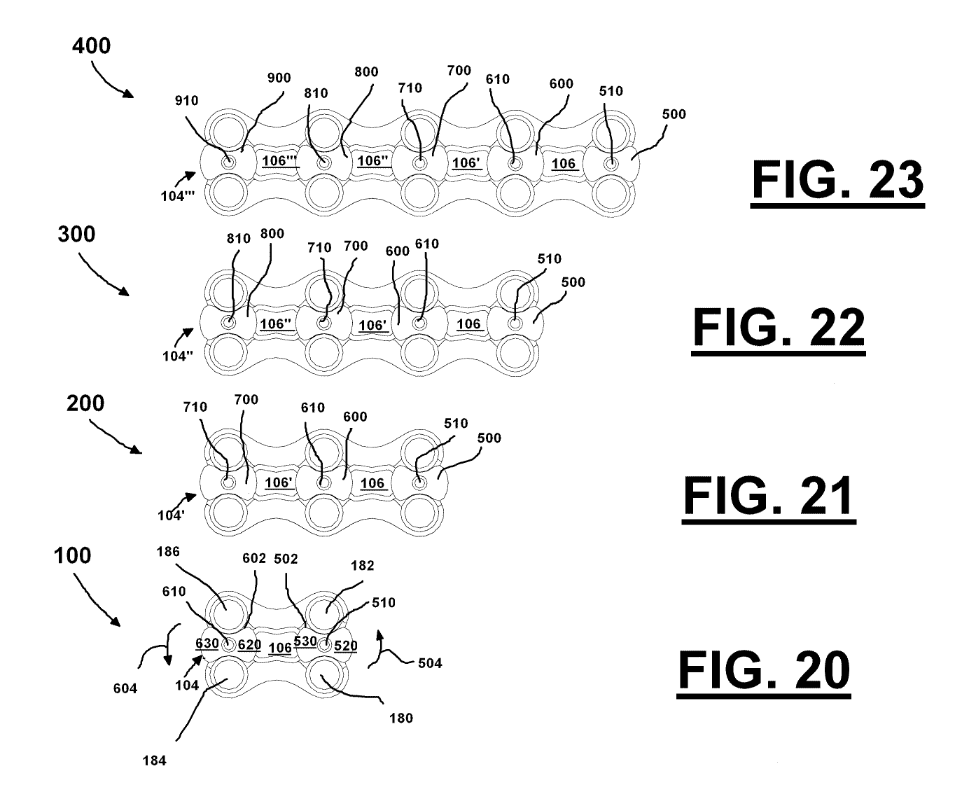

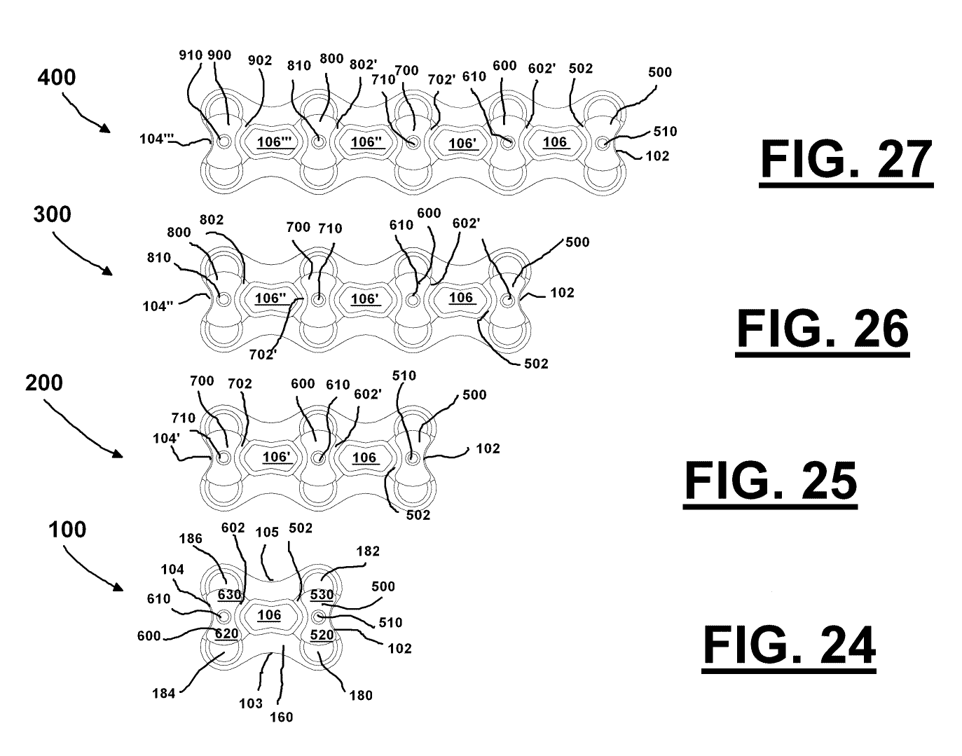

FIGS. 1 and 24 are top views of plate 100 with locking covers 500 and 600 rotated such that they are in a locked position. FIG. 20 is a top view showing locking covers 500 and 600 in an unlocked position. FIG. 15 is a top view where all locking covers have been removed. As shown in FIGS. 1, 15, 20, and 24, locking covers in the unlocked position extend outside of the body of plate 100. For example, first side 520 of locking cover 500 extends outside of cutout 105; and second side 530 extends into cutout 106. As another example, second side 630 of locking cover 600 extends outside of cutout 104; and first side 620 extends into cutout 106. However, in the locked positions neither of these covers extends outside of the body of plate 100. Because of such extension or extensions in the unlocked position, X-rays could determine whether either of these locking covers are in the unlocked position because at least part of the locking cover would show up in the X-ray. Accordingly, such property provides a convenient method of confirming that the locking covers are in a locked position.

In one embodiment screws can be used to attach plate 100 to upper and lower vertebrae. In one embodiment screws can be bone screws. In one embodiment openings 180, 182, 184, and 186 can include recessed areas which allow the heads of screws to be flush with the top surface of plate 100.

In one embodiment pivot locations 510 and 610 are large enough to be used as a cervical pin holder. Plate 100 can be temporarily fixed by two cervical pins positioned at points 510 and 610—which locations are also the pivot points for locking covers 500 and 600. In one embodiment the method of positioning and holding fixed plate 100 by installing cervical pins first in openings 510 and 610 through the vertebrae, drilling holes in the vertebrae through openings 180, 182, 184, and 186 into the vertebrae, installing screws in the vertebra

In one embodiment a pair of bone screw holes is used for each vertebra to be fused. For example, a plate for a one level fusion would have two pairs of bone screw holes—one in each of the vertebra being fused. As another example, for a two level fusion each of the three vertebra would have a pair of screw holes. For each of these pairs of screw holes the cervical plate can have a pair of openings, and a locking cover which is located in the middle of the paired openings.

In one embodiment, the bottom surface 150 of plate 100, preferably has a textured, roughened, and/or porous surface (shown is a ribbed or knurled surface). In one embodiment bottom surface 150 can be coated with, impregnated with, or comprise fusion promoting substances (such as bone morphogenetic proteins) so as to encourage the growth of bone from vertebrae to vertebrae. In one embodiment plate 100 can comprise at least in part a resorbable material which can further be impregnated with the bone growth material so that as plate 100 is resorbed by the body of the patient, the bone growth material is released, thus acting as a time release mechanism.

As shown in FIGS. 16-18, 21-23, and 25-27, in alternative embodiments, fusion plates 200, 300, 400 for fusing more than two levels could have additional screw openings corresponding to additional paired bone screw holes. In one embodiment additional locking covers can be provided for each of the paired screw openings. In one embodiment the locking covers are pivotally connected to the plate, and at the point of rotation an opening for a positioning pin can be found.

FIG. 16 is a top view of an alternative fusion plate 200 for two levels where the locking covers have been removed for clarity. FIG. 21 is a top view of fusion plate 200 for two levels showing the locking covers (500, 600, 700) in an unlocked position. FIG. 25 is a top view of fusion plate 200 for two levels now showing the locking covers (500, 600, 700) in a locked position. In the unlocked position it can be noticed that at least part of the locking covers will be visible though an opening (e.g., 106 or 106′) and/or through a cutout (e.g., 102, 104′).

FIG. 17 is a top view of an alternative fusion plate 300 for three levels where the locking covers have been removed for clarity. FIG. 22 is a top view of fusion plate 300 for three levels showing the locking covers (500, 600, 700, 800) in an unlocked position. FIG. 26 is a top view of fusion plate 300 now showing the locking covers (500, 600, 700, 800) in a locked position. In the unlocked position it can be noticed that at least part of the locking covers will be visible though an opening (e.g., 106, 106′, 106″) and/or through a cutout (e.g., 102, 104″).

FIG. 18 is a top view of an alternative fusion plate 400 for four levels where the locking covers have been removed for clarity. FIG. 23 is a top view of fusion plate 400 for four levels showing the locking covers (500, 600, 700, 800, 900) in an unlocked position. FIG. 27 is a top view of fusion plate 400 for four levels now showing the locking covers (500, 600, 700, 800, 900) in a locked position. In the unlocked position it can be noticed that at least part of the locking covers will be visible though an opening (e.g., 106, 106′, 106″, 106′″) and/or through a cutout (e.g., 102, 104″′).

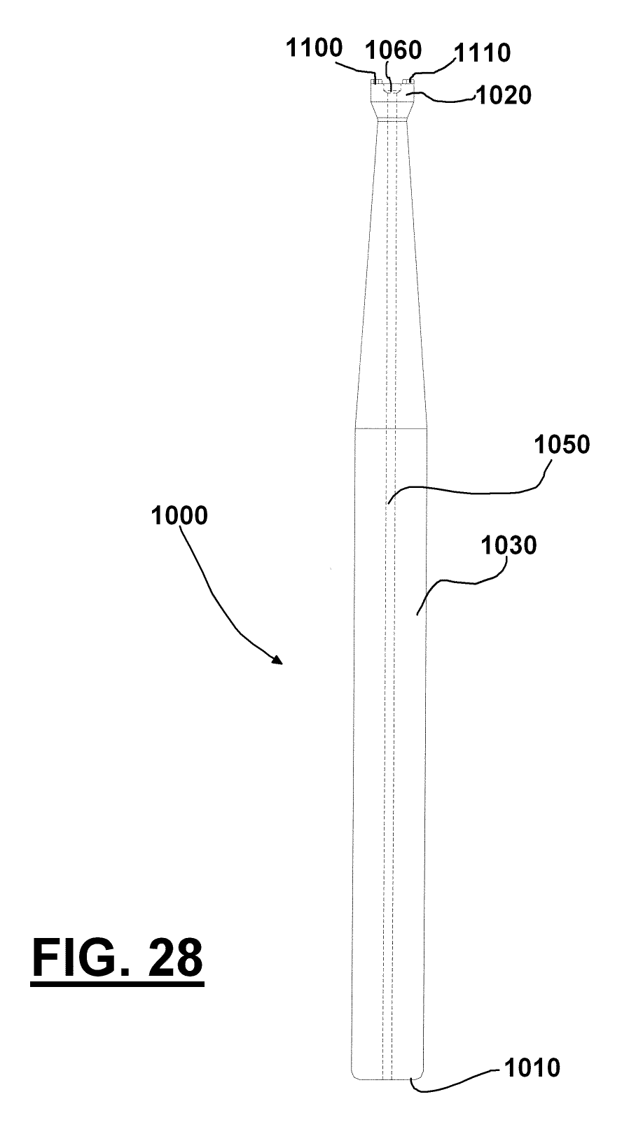

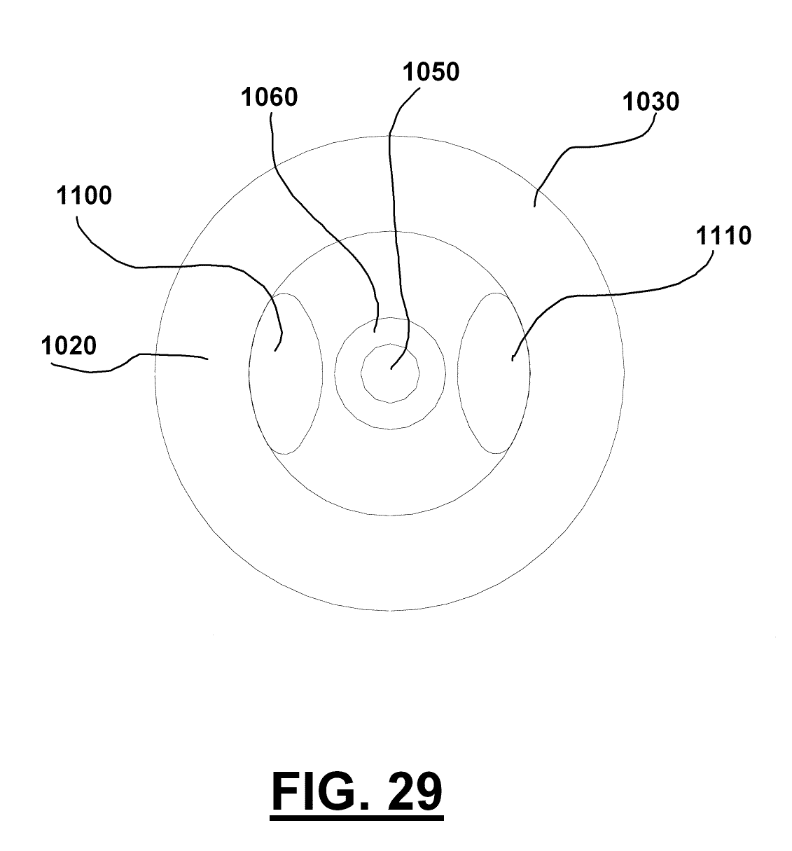

FIG. 28 is a side view of a installation tool 1000 which can be used to lock and unlock the locking covers (e.g., 500, 600). FIG. 29 is a bottom view of the tip or second end 1020 of the installation tool 1000. Installation tool 1000 can include first end 1010, second end 1020, and body 1030. On second end or tip can be included first and second projections 1100, 1110. First and second projections 1100 and 1110 can fit around the middle portion of the locking covers such as locking cover 500 (and between first and second ends 520, 530 of locking cover 500) such on opposite sides of the pivot point 510 of locking cover.

Projections 1100 and 1110 can have a shape which fits the locking covers (e.g., 500,600) tool 1000 turns. Because locking covers (e.g., 500,600) have an “hour glass” shape, projections 1100 and 1110 can have curved internal surfaces to match the hour glass shape of projections. Projections 1100 and 1110 are believed to be self centering over locking covers (because the shape of locking covers match the interior shape of projections). However, various other mechanisms can be used to assist in positioning and/or centering tool 1100 over a locking cover.

Shown in this embodiment is a recessed area 1060 which is designed to accept the raised area around the pivot points of any locking cover (e.g., pivot point 510 of locking cover 500), which centers tool 1000 around the pivot point and the locking cover. Also shown in this embodiment is central opening 1050 extending from first end 1010 to second end 1020 of body 1030. Central opening 1050 allows the used of a centering tool such as a centering rod or wire to be placed through body 1030 and enter the pivot point (e.g., pivot point 510 of locking cover 500) of the locking cover to be locked or unlocked to center tool 1000 over such locking cover. Alternatively, recessed area 1060 and central opening can be constructed to accept a cervical positioning pin which was placed in the pivot point (e.g., pivot point 510 of locking cover 500) so that the positioning pin centers tool 1000. Also alternatively, a cervical positioning pin can be installed in a cervical plate while tool 1000 is installed on a locking cover (e.g., pivot point 510 of locking cover 500).

In an alternative embodiment a middle projection or pointer can replace recessed area 1060 where the middle projection or pointer would enter the pivot point of the locking cover (e.g., pivot point 510 of locking cover 500) which would center the tool over locking cover. In another alternative embodiment tips 1100 and 1110 can be increased in length so that the raised portion of the pivot point (e.g., pivot point 510 of locking cover 500) of a locking cover is less than the length of the projections—otherwise, if such was not the case, the raised portion interfere with the projections interlocking with the locking cover to be locked or unlocked.

In use projections 1100 and 1110 of tool 1000 are placed over a locking cover to be locked and turned (causing the locking cover to turn) to a locked position (locked positions are shown in FIGS. 24 through 27). Additionally, projections 1100 and 1110 of tool 1000 can be placed over a locking cover to be unlocked and turned (causing the locking cover to turn) to an unlocked position (unlocked positions are shown in FIGS. 20 through 23).

### Surgical Method for a Single Level Fusion

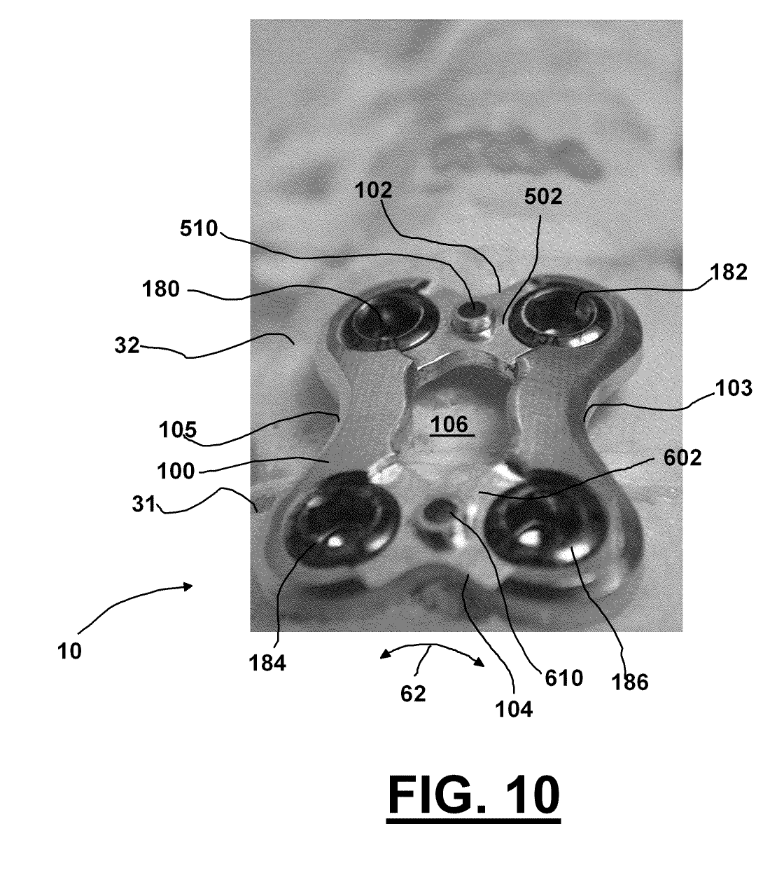

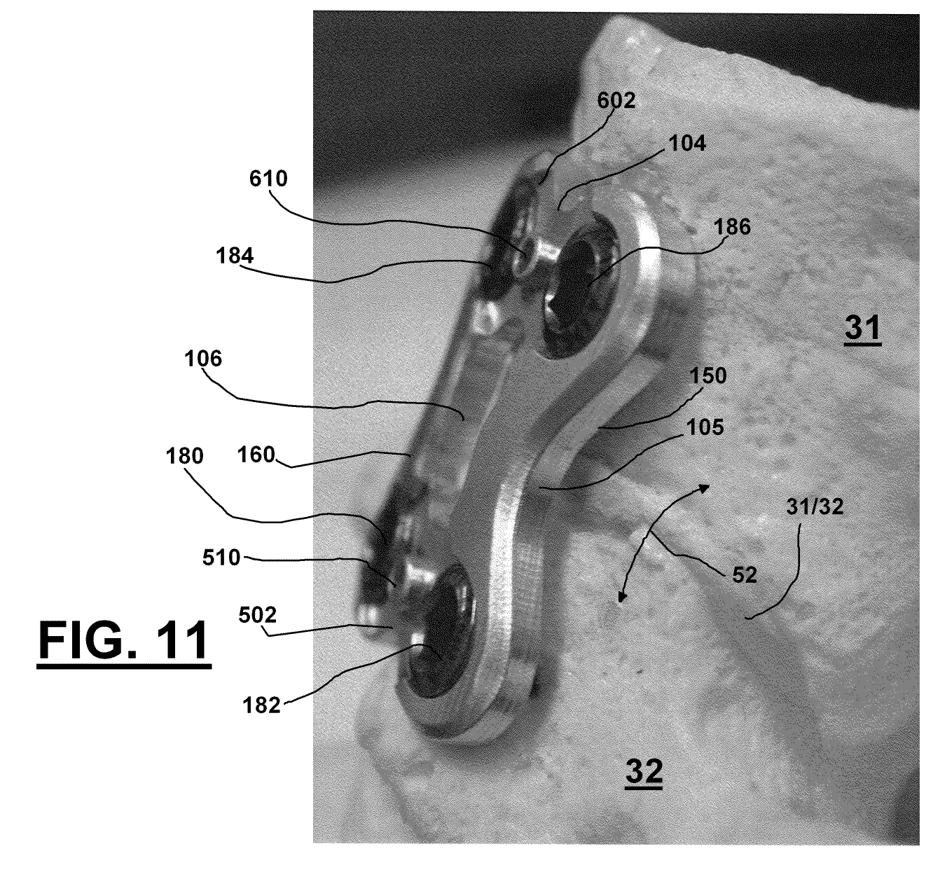

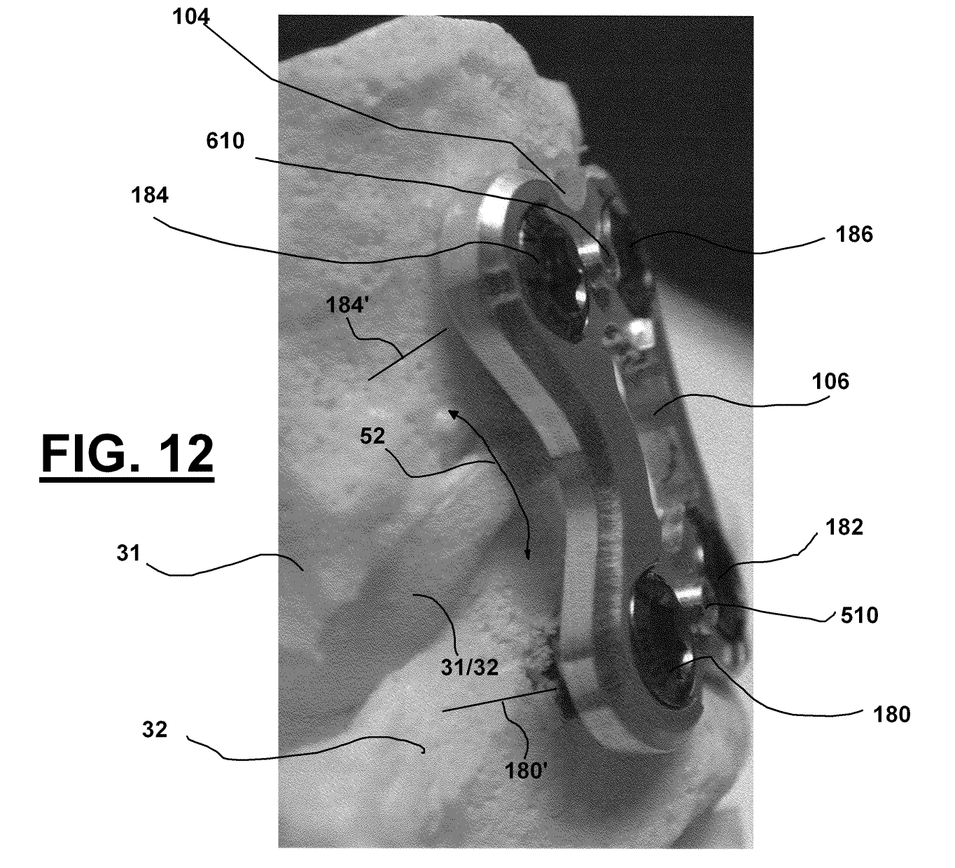

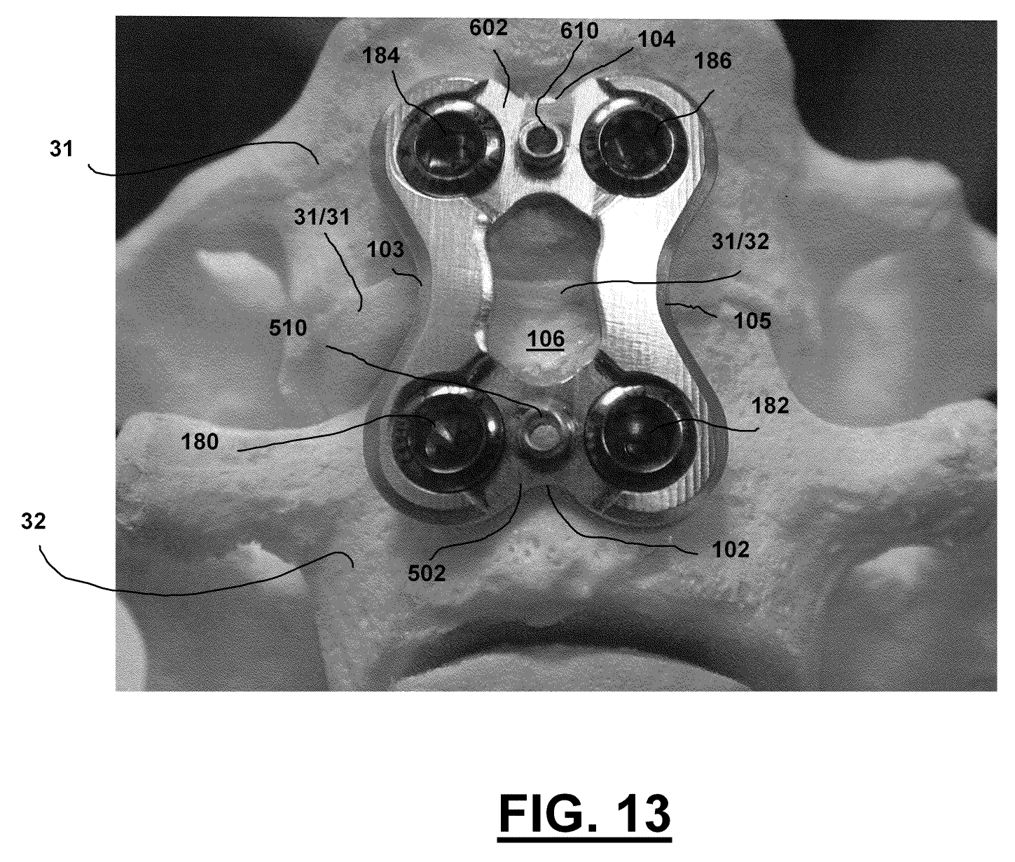

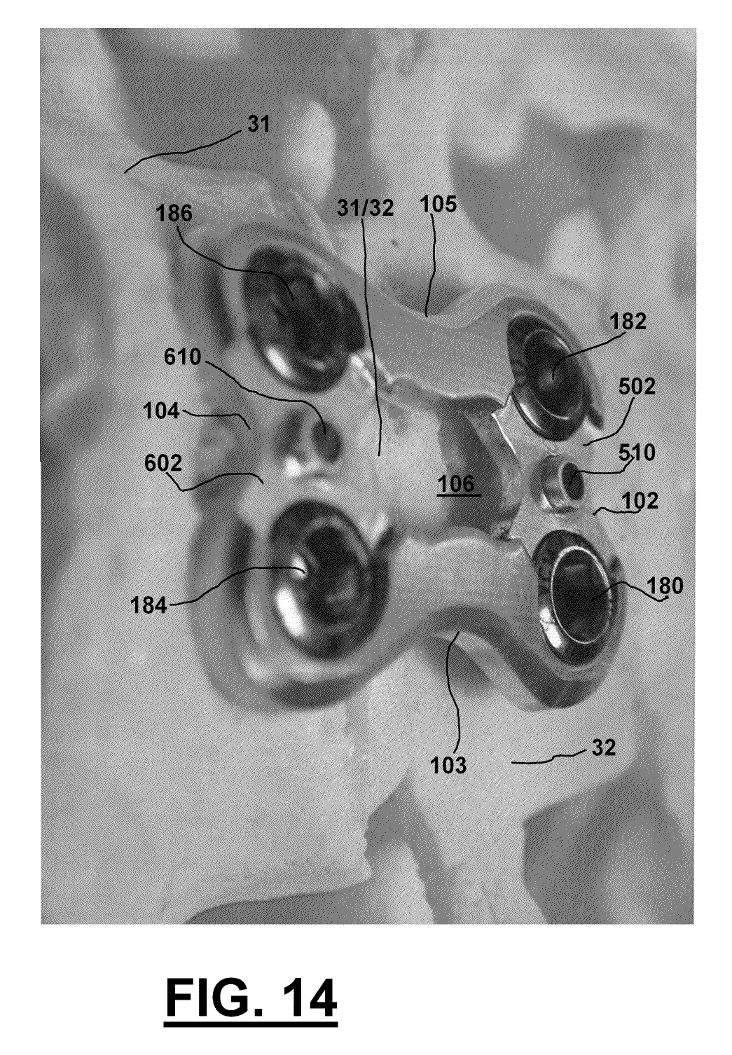

FIGS. 9 through 14 show plate 100 affixed to cervical vertebrae 31 and 32. In these figures locking covers 500 and 600 have been removed to be able to view the bone screws in openings 180,182,184, and 186.

When the cervical fusion apparatus 10 is implanted in a patient, a surgeon dissects the patient's throat below the patient's chin to access the cervical spinal column 20 of the patient. Optionally, the throat dissection may be done asymmetrically with respect to the throat to minimize invasion of the throat while positioning the cervical fusion plate 100 in the desired relationship with the cervical spinal column 20. The surgeon then inserts the cervical fusion plate 100 into the throat dissection and maneuvers the cervical fusion plate 100 around the larynx and other throat structures to a position near the spinal column 20. The size or shape of the cervical fusion plate 100 may be chosen to minimize damage or irritation to the patient's throat during implantation while maintaining strength to retain the vertebrae (e.g., 31 and 32) in the desired relationship after implantation.

Once the cervical fusion plate 100 is located near the spinal column 20, the surgeon positions the cervical fusion plate 100 on the cervical spinal column 20. The surgeon contacts a portion of the anterior surface of a first vertebra (e.g., 31) and a side portion of the anterior surface of a second vertebra (e.g., 32) with the cervical fusion plate 100.

The surgeon uses cervical positioning pins in openings 510 and 520 to hold the cervical fusion plate 100 in engagement with the vertebrae (e.g., 31 and 32) while drilling or tapping reception holes in the vertebrae to receive the fasteners or bone screws. These reception holes should match the openings for the fasteners (e.g., 180, 182, 184, and 186) for precise implantation of the cervical fusion apparatus 100. Alternately, the vertebrae (e.g., 31 and 32) could be prepared with such reception holes before the cervical fusion plate 100 becomes engaged with the vertebrae. However, if the fasteners are self-tapping, the surgeon need not prepare reception holes but instead might drill small pilot holes or even just initially penetrate the anterior surface of the vertebrae directly with the fasteners to be installed.

Once the cervical fusion plate 100 has been positioned as desired and the vertebrae (e.g., 31 and 32) have been prepared as needed to accept the fasteners, the surgeon inserts the fasteners through the holes (180 and 182) and into the first vertebra (e.g., 31). The surgeon also inserts fasteners through a second fastener openings (184 and 186) and into the second vertebra (e.g., 32). Once these fasteners have been tightened and anchored as desired, the cervical fusion apparatus 100 is operative to retain the engaged vertebrae (e.g., 31 and 32) in the desired spatial relationship.

The surgeon can use tool 1000 to lock locking covers 500 and 600 over the fasteners. Projections 1100 and 1110 connect to locking cover 500 and can be turned to a locked position. Projections 1100 and 1110 connect to locking cover 600 and can be turned to a locked position. Locking covers 500 and 600 now resist loosening of the fasteners in openings 180, 182, 184, and 186.

When all of the desired fasteners have been implanted to the surgeon's satisfaction, all tools are removed from the throat dissection and the patient's throat is closed in a known manner.

The method and apparatus of certain embodiments of the present invention, when compared with other apparatus and methods, may have the advantages of: being useful in a minimally invasive surgical procedure, allowing the patient's throat structures to remain largely in place during the operation, being usable in a timely and efficient manner, and being more economical to manufacture and use. Such advantages are particularly worthy of incorporating into the design, manufacture, and operation of a cervical fusion apparatus 10. In addition, the present invention may provide other advantages which have not yet been discovered.

Some of the perceived advantages of fusion plates 100, 200, 300, and 400 are as follows:

(A) Plate profile and graft window allow for good visibility of graft;

(B) Pre-curved plate reduces the amount of possible bending needed during the surgical procedure;

(C) Due to the location of anti-migration locking covers allows for a reduction of the risk of damage to the locking covers and/or too large of play between locking covers and screws being constrained. That is, if the pivot points were moved and the plate bent, an excessive gap could occur between the locking cover and the screw being held by the locking cover;

(D) The pivot point of a locking cover also being used as an insertion point for a cervical pin holder, allows for the use of a temporary plate holding pin during initial installation; and

(E) Easy to use locking covers allows surgeon to easily remove existing 4 millimeter bone screws and install 4.5 millimeter recovery screws if necessary for a fusion.

Some non-limiting examples of plate features include a precurved plate body in both the traverse and sagittal planes; graft window; capability of accepting fixed and variable angle screws; uses 4 millimeter bone screws and 4.5 millimeter recovery screws; easy to use anti-migration locking covers with a hole through the cover and plate for use of a plate holding pin.

While aspects of the present invention have been particularly shown and described with reference to the preferred embodiment above, it will be understood by those of ordinary skill in the art that various additional embodiments may be contemplated without departing from the spirit and scope of the present invention. For example, the elements of the present invention could be made of any variety or combinations of materials, though preferably all chosen materials are biocompatible; the fasteners could be angled differently than described; the fasteners could be inserted in a different order than described, the cervical fusion plate 100 could include various contours, apertures, or other shaping providing advantages in strength, weight, infection-resistance, cost, or the like; any desired number of addition/different vertebrae could be engaged with the cervical fusion plate 100; or the engaged vertebrae need not be adjacent.

The following is a list of reference numerals:

LIST FOR REFERENCE NUMERALS

| (Ref No.) | (Description) |
|---|---|
| 10 | cervical fusion apparatus |
| 15 | patient |
| 20 | cervical spinal column |
| 30 | plurality of vertebrae 30 |
| 31 | vertebra |
| 32 | vertebra |
| 33 | vertebra |
| 34 | vertebra |
| 50 | line or axis |
| 52 | arrow |
| 60 | line or axis |
| 62 | arrow |
| 100 | cervical fusion plate |
| 102 | portion |
| 103 | portion |
| 104 | portion |
| 105 | portion |
| 110 | viewing window |
| 150 | bottom surface |
| 160 | upper surface |
| 180 | screw opening |
| 182 | screw opening |
| 184 | screw opening |
| 186 | screw opening |
| 200 | cervical fusion plate for two level fusion |
| | (three adjacent vertebrae) |
| 300 | cervical fusion plate for three level fusion |
| | (four adjacent vertebrae) |
| 400 | cervical fusion plate for four level fusion |
| | (five adjacent vertebrae). |
| 500 | locking cover |
| 502 | recessed area |
| 504 | arrow |
| 510 | pivot point or opening for positioning pin |
| 520 | first end |
| 530 | second end |
| 600 | locking cover |
| 602 | recessed area |
| 604 | arrow |
| 610 | pivot point or opening for positioning pin |
| 620 | first end |
| 630 | second end |
| 632 | arrow |
| 634 | arrow |
| 700 | locking cover |
| 702 | recessed area |
| 710 | pivot point or opening for positioning pin |
| 720 | first end |
| 730 | second end |
| 800 | locking cover |
| 802 | recessed area |
| 810 | pivot point or opening for positioning pin |
| 820 | first end |
| 830 | second end |
| 900 | locking cover |
| 902 | recessed area |
| 910 | pivot point or opening for positioning pin |
| 920 | first end |
| 930 | second end |
| 1000 | installation tool |
| 1010 | first end |
| 1020 | second end or tip |
| 1030 | body |
| 1050 | central bore |
| 1060 | recessed area |
| 1100 | first projection |
| 1110 | second projection |

All measurements disclosed herein are at standard temperature and pressure, at sea level on Earth, unless indicated otherwise. All materials used or intended to be used in a human being are biocompatible, unless indicated otherwise.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only; the scope of the present invention is to be limited only by the following claims.

1. An anterior cervical plate for engaging at least two vertebral bodies of a human cervical spine along the anterior aspect of the spine, the plate having a lower surface for contacting the cervical vertebral bodies and an upper surface opposite that of the lower surface, the plate having a plurality of bone screw receiving holes extending through the plate from the upper surface to the lower surface, at least a first and second of said bone screw receiving holes being associated with a first of the cervical vertebral bodies, and at least one locking cover pivotally connected to the plate and having locked and unlocked positions, the pivot point of the locking cover being a cervical pin positioning opening.

2. The plate of claim 1, wherein the locking cover capable of locking at least two bone screws in the at least first and second bone screw receiving holes, the pivot point being located on a transverse line passing through the central longitudinal axes of the first and second bone screw receiving holes.

3. The plate of claim 1, wherein the plate has a length longer than the width, and the lower surface has a first concave curvature parallel to the width.

4. The plate of claim 1, wherein the lower surface of the plate has a second concave curvature parallel to the length.

5. The plate of claim 1, wherein the plate has a length sufficient to span at least three consecutive vertebral bodies.

6. The plate of claim 1, wherein the lower surface of the plate has a complex concave curvature configured to mate with at least two vertebral bodies, and each bone screw receiving hole has a longitudinal axis that is generally perpendicular to the lower surface at the location of said bone screw receiving hole and each of said bone screw receiving holes is formed to retain a respective bone screw in a position in which the longitudinal axis of said respective bone screw is aligned with the longitudinal axis of the bone screw receiving hole.

7. An anterior cervical plate for engaging at least two vertebral bodies of a human cervical spine along the anterior aspect of the spine, the plate having a length sufficient to span at least two adjacent cervical vertebral bodies, the plate having a lower surface for contacting the cervical vertebrae and an upper surface opposite to the lower surface, the plate having a plurality of bone screw receiving holes extending through the plate from the upper surface to the lower surface, at least a first and second of the bone screw receiving holes being associated with a first of the cervical vertebral bodies, and a recess which includes a pivotally connected locking cover and at the pivot point being located an opening extending from the upper to lower surface, the locking cover being movable from at least a first position to a second position through a pivoting action, the first position enabling insertion of bone screws into each of the first and second bone screw receiving holes, and the second position causing the locking cover to bear against at least a portion of at least two of the bone screws in the first and second bone screw receiving holes, the opening at the pivot point being a cervical pin positioning opening.

8. An anterior cervical plating system for engaging at least three vertebral bodies of a human cervical spine along the anterior aspect of the spine, the system comprising:
a plate having a generally rectangular configuration with a first end, a second end, sides, and a length sufficient to span at least two adjacent cervical vertebral bodies, the plate having:
rounded edges at each corner of the generally rectangular configuration and having rounded lobes on the sides between the first and second ends;
a lower surface for contacting the cervical vertebral bodies and an upper surface opposite to the lower surface;
a bi-concave curvature for conforming to the anterior aspect of the cervical spine in lordosis, the bi-concave curvature having a longitudinal concave curvature along the longitudinal axis of the plate and a transverse concave curvature along the transverse axis of the plate;
a plurality of bone screw receiving holes extending through the plate from the upper surface to the lower surface, a respective one of the plurality of bone screw receiving holes located at each of the rounded edges such that the plate has a first pair of the bone screw receiving holes located at the first end of the plate and corresponding to a first of the adjacent vertebral bodies, a second pair of the bone screw receiving holes corresponding to a second of the adjacent vertebral bodies, and a third pair of the bone screw receiving holes corresponding to a third of the adjacent vertebral bodies;
a locking cover pivotally connected to the plate and including a cervical pin positioning opening at the pivot point; and
the locking cover being movable between a first unlocked position and to bear against at least a portion of the bone screw in the bone screw receiving holes when moved to a second locked position.

9. A method of implanting a cervical fusion plate, having at least two fastener openings, into a patient, the method comprising the steps of: dissecting a throat of the patient; providing access through the throat dissection to a spinal column of the patient; inserting the fusion plate into the throat dissection; positioning the fusion plate on the spinal column; the fusion plate comprising engaging at least two vertebral bodies of a human cervical spine along the anterior aspect of the spine, the plate having a lower surface for contacting the cervical vertebral bodies and an upper surface opposite that of the lower surface, the plate having a plurality of bone screw receiving holes extending through the plate from the upper surface to the lower surface, at least a first and second of said bone screw receiving holes being associated with a first of the cervical vertebral bodies, and at least one locking cover pivotally connected to the plate and having locked and unlocked positions, the pivot point of the locking cover being a cervical pin positioning opening, and placing cervical positioning pins in the positioning openings for holding the position of the plate relative to the spine.

10. (canceled)
